(19) United States
(12) Patent Application Publication
(10) Pub. No.: US 2021/0267852 A1
(43) Pub. Date: Sep. 2, 2021

(54) Methods For Repairing Skin Using Nanofibers

(71) Applicant: ELC Management LLC, Melville, NY (US)

(72) Inventors: Daniela BRATESCU, Northport, NY (US); Isaac David COHEN, Brooklyn, NY (US); Zhihan ZHOU, Metuchen, NJ (US); Peter A. LANDA, Springfield, NJ (US); Milan Franz SOJKA, Coram, NY (US)

(21) Appl. No.: 17/187,883

(22) Filed: Mar. 1, 2021

Related U.S. Application Data
(60) Provisional application No. 62/984,179, filed on Mar. 2, 2020.

(51) Int. Cl. A61K 8/02; A61Q 19/08; A61K 8/81; A61K 8/73; A61K 8/86; A45D 44/00

(52) U.S. Cl. CPC A61K 8/027; A61K 8/0212; A61K 8/0208; A61Q 19/08; A61K 8/8176; A61K 2800/884; A61K 8/86; A45D 44/002; A61K 2800/30; A61K 2800/41; A61K 8/735

(57) ABSTRACT

The present invention provides novel methods for repairing or reducing skin changes as well as systems and cosmetic compositions thereof. A method for reducing the appearance of dark circles, puffiness, or sagginess in the periorbital area of a subject's skin, comprising topically applying a composition comprising electrospun fibers to the subject's skin in need thereof is provided. The method further causes firming or tightening of the skin around the periorbital area of the subject. The composition provided herein is substantially free of active ingredients or devoid of active ingredients.

### FIELD

The present invention generally relates to methods, systems, and cosmetic compositions for repairing and reducing skin defects using polymeric fibers, including microfibers and nanofibers.

### BACKGROUND

Aging, exposure to adverse environmental factors, pollutants, lack of good nutrition, fatigue, can affect the visual appearance, physical properties, or physiological functions of the skin. These factors may create a visually undesirable appearance to the skin. Notable changes including dilation of the veins, bags under the eyes, dark circles under the eyes, and swelling around the eyes may occur. Skin aging or environmental damage to the skin also causes fine lines and wrinkles, loss of elasticity, loss of stiffness, loss of uniformity of color or tone, rough surface texture, age spots, and a decrease in moisture content. Many of such changes in the appearance and function of the skin are caused by changes in the outer epidermal layer of the skin, while other changes are caused by changes in the lower dermis.

It is therefore an object of the present invention to provide novel methods for repairing or reducing skin changes, as well as systems and cosmetic compositions thereof.

### SUMMARY

A method of the reducing appearance of puffiness in periorbital area of a subject's skin, comprising topically applying a composition comprising electrospun fibers to the subject's skin in need thereof. The method further causing firming or tightening of the skin around the periorbital area of the subject, wherein the composition is substantially free of active ingredients or is devoid of active ingredients. The composition further comprises cosmetically acceptable carriers, an anti-oxidant or moisturizers or softeners or preservatives. The composition is a patch or a mask with a thickness (areal density) ranging from about 0.1 to about 50 gsm and is made of electrospun fibers that comprise hydrophilic polymers.

A method for improving the visual appearance of a subject's skin, comprising providing a subject a solution to wet the skin, and administering a composition topically to the skin, wherein the composition comprises electrospun hydrophilic fibers is also provided, wherein the composition is substantially free of active ingredients or is devoid of active ingredients. The method further includes the step where the skin is wetted by spraying a solution or through a spray and the method improves dilation of the veins, bags under the eyes, dark circles under the eyes, and swelling around the eyes, lifting, firming, smoothing of the skin, fine lines, wrinkles, or tightening of the skin. Upon wetting, the composition dissolves in less than two minutes of wetting.

A cosmetic patch comprising hydrophilic electrospun fibers, wherein the patch is substantially free of active ingredients is also provided. The patch further comprises one or more adhesive layers and is devoid of the active ingredients.

A kit comprising a pre-wetting solution, and a cosmetic patch comprising hydrophilic electrospun polymeric fibers. The patch may be a mask or film.

### BRIEF DESCRIPTION OF THE DRAWINGS

FIG. 1 shows the results of live grading for under-eye puffiness using nanofiber patches.

FIG. 2 shows the results of live grading for under-eye puffiness using nanofiber patches and caffeine.

FIG. 3 shows the results of pullulan on MatTek skin.

### DETAILED DESCRIPTION

To facilitate an understanding of this invention, several terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

The terms “active ingredient” or “active agent” or “cosmetic agent” means a cosmetic agent or an ingredient that is utilized to deliver a benefit to the skin. “Active ingredient” or “active agent” or “cosmetic agent” would cause to, drive a change in the subject's skin or deliver the benefits under consideration, thus, aid in accomplishing a desired, expected, or intended result. It is not intended that the “active ingredient” or “active agent” or “cosmetic agent” according to the present invention include cosmetically acceptable excipients or carriers that may be present in a composition/formulation. Further, cosmetic agents when present in free form or formulated as powders, emulsions etc., can be considered as an active ingredient according to the present invention.

The terms “prevent” and “preventing” include the prevention of the recurrence, spread or onset of a skin or hair condition. It is not intended that the present invention be limited to complete prevention.

The term “subject” refers to any mammal, preferably a human.

The term “topical” refers to the administration of an agent or agents (e.g., cosmetic, vitamin, etc.) on the skin.

The terms “transdermal” or “topical” refers to the delivery of an agent (e.g., cosmetic, dermatological, vitamin, etc.) through the skin (e.g., so that at least some portion of the population of particles reaches underlying layers of the skin).

The term “hydrophilic” refers to the physical property of a molecule that is able to transiently associate with water, i.e., bond with water via hydrogen bonding.

The term “solvent” refers to a liquid, solid, or gaseous solute generating a solution.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The term “substantially free of active ingredients” refers to, when used, cosmetic active ingredients or cosmetic agents as defined herein (such as in free form or powders etc.) are present in amounts less than about 0.00001% by the relative weight of the composition.

The term “devoid of active ingredients” refers to, when used, cosmetic active ingredients or cosmetic agents as defined herein (such as in free form or powders etc.) are absent in the composition/method or the composition is free of cosmetic active ingredients, while the composition may include carriers, excipients, softeners or preservatives, as needed. However, electronspun polymeric fibers such as PVP, pullulan or HA, among others, are considered to be outside of the definition of active ingredients as defined herein.

The term microfiber refers to fibers having a diameter of greater than 1000 nm.

The term nanofiber refers to fibers having a diameter of less than 1000 nm.

Except in operating and comparative examples, or where otherwise explicitly indicated, all numbers in this description indicating amounts or ratios of material or conditions of reaction, physical properties of materials and/or use are to be understood as modified by the word “about”. All amounts are presented as percentages by weight of the final cosmetic agent unless otherwise specified.

The present invention relates to methods for repairing skin using polymeric fibers, including microfibers or nanofibers. In some embodiments, the present invention relates to compositions for repairing the skin. Measurable changes appear in the skin as the skin ages or endure environmental or age-related insult. Such insults cause a general reduction in cellular and tissue vitality, reduction in cell replication rates, reduced cutaneous blood flow, reduced moisture content, errors in structure and function, alterations in biochemical pathways, and reduction of skin's ability to remodel and repair itself.

As a non-limiting example, human skin around the periorbital area (i.e., around the eyes) is thin and delicate. The periorbital area is webbed with tiny capillaries, and blood sometimes leaks from these capillaries causing an appearance of dark circles under the eye. Other known causes of dark under-eye circles include UV exposure (e.g., exposure to the sun can increase natural melanin levels and draws the melanin to the surface of the skin, making it darker), ageing (e.g., with age, the skin around the eyes can become even thinner which makes dark under-eye circles become more pronounced), fatigue (being tired can make skin paler which makes dark circles look darker), allergies (e.g., allergic reactions can cause smudges in the under-eye area and conditions that causes a person to rub their eyes can make dark circles worse because scratching or rubbing can darken the skin), pregnancy or menstruation (e.g., skin becomes pale during pregnancy and menstruation which makes dark circles look darker), and inadequate nutrition.

As another non-limiting example, a condition where the skin under the eyes swells and becomes visually undesirable is called puffy eyes. Puffy eyes can be caused by several factors including, increased vascularization, leaky capillaries, thinning/slackening skin which can fill up with more fluid, loss of the fat pad under the eye which can contribute to under-eye bags, and allergies, dust, and pollutants which can trigger a release of chemicals, thereby, swelling the tissue around the eyes.

Methods for treating or repairing, for example, the puffy eyes or dark circles include stimulation of the dermis or epidermis with cosmetic active ingredients. The use of such agents may renew skin cell rate and cause basal cell division. Several approaches have been utilized to prevent, reduce or treat damage to the skin, especially eyes caused by environmental factors, chemicals, pollutants, or aging. Most of these approaches to-date involve delivering one or more agents to act on the skin to cause an effect. In many approaches, polymeric materials are utilized to effectuate the delivery of such agents. Examples of such agents include retinoids to stimulate collagen, tools to stimulate epidermis renewal, such as films, or patches impregnated or carrying one or more active agents within the patch. Most such applications require a period of time to several weeks to sometimes months for the desired effect to become visible. The Applicants of the present invention discovered that the physical properties of the electrospun hydrophilic nano or micro polymeric fibers are able to effect, prevent, or treat defects on a subject's skin even without the presence of cosmetic active ingredients or when cosmetic active ingredients are substantially absent. One or more hydrophilic polymeric fibers may be utilized in electrospinning resulting in the electronspun fibers of the present invention.

In particular, the methods and compositions disclosed herein may be used to prevent or treat changes to the skin, such as dilation of the veins, bags under the eyes, dark circles under the eyes, and swelling around the eyes, fine lines and wrinkles, increase in firmness (firming), or tightening of the facial skin, reduce the loss of elasticity, wrinkles, reduce the loss of stiffness or uniformity of color or tone, rough surface texture, age spots, and a decrease in moisture content in the skin, smoothing of the skin, fine lines and/or wrinkles, and lifting and firming of the skin. Particularly, the present invention relates to methods for repairing or reducing dilation of the veins, bags under the eyes, dark circles under the eyes, swelling around the eyes, fine lines and wrinkles, loss of elasticity, increase tightness or firmness, reduce the loss of stiffness or uniformity of color or tone, rough surface texture, age spots, and a decrease in moisture content in the facial skin, particularly around the eyes.

According to an aspect of the present invention, compositions and methods to treat the appearance of the skin are provided. In one embodiment, the compositions and methods are substantially free of active ingredients or cosmetic agents. In other embodiments, such compositions and methods are devoid of the cosmetic active ingredients or cosmetic agents.

In some embodiments, the present invention contemplates a solid, or a semisolid composition such as, for example, a patch or a film or a mask that may partially or fully dissolve or disappear (i.e., become invisible to the naked eye) for use on the skin.

The present application describes a method of repairing or treating skin or reducing skin defects and improving the visual appearance of the skin, especially the skin surrounding and under the eyes using polymeric fibers, including microfibers or nanofibers. Applicants of the present invention surprisingly discovered that when nanofibers were utilized, without limiting, as a patch or a film, on the skin or under the eyes, and when such nanofibers are substantially free of active ingredients or cosmetic agents, skin aging and skin damage were significantly reduced or improved within a short period of time. In preferred embodiments, the Applicants discovered that when nanofibers were utilized, without limiting, as a patch or a film, as a composition on the skin under the eyes or on the skin, and when the composition is devoid of active ingredients or cosmetic agents, skin aging and skin damage were significantly reduced or improved within a short period of time.

Electrospinning is a versatile technique utilized for creating fibrous scaffolds of nanomaterials. Polymers and/or polymeric materials, among other materials may be utilized in such process and the nanomaterials are formed by deposition, resulting in an interconnected three-dimensional network or scaffold. A skilled person in the art would readily be able understand and utilize the process of electrospinning.

In one embodiment, the present invention contemplates a composition comprising a dissolving or a disappearing patch made of polymeric electrospun micro or nanofibers for use on the skin. The composition according to the present invention may be a solid, semi-solid, semi-liquid composition, such as a patch or mask. The patch may be in form of a film. It is contemplated that the composition, for example, the patch of polymeric electrospun micro or nanofibers is substantially free of or devoid of active ingredient or agent and yet may comprise cosmetically acceptable carriers, excipients, or softeners. In preferred embodiments, the composition, for example, is the patch of micro or nanofibers that is substantially free of or devoid of the active ingredients or cosmetic agents.

In one embodiment, the composition, such as a film or patch comprises one or more hydrophilic or hydrophobic polymer fibers, or mixtures thereof. The polymer fiber is porous and mechanically strong for application on the skin without tearing. Structural material for the patch or film is chosen in order to achieve desired dissolution rate on the skin. By dissolution, the fibers would become invisible to the naked eye upon or subsequent to the topical application. In some embodiments, the dissolution rate ranges from about 5 seconds, 10 seconds, 20 seconds, 1 minute, 2 minutes 5 minutes, or 10 minutes. Any subranges are considered to be within the scope of the present invention. Hydrophilic polymeric fiber includes, without limiting, poly (ethylene glycol), poly(propylene glycol), poly(vinyl alcohol), polypyrrolidone or polyvinylpyrrolidone (PVP), and the biodegradable polyactive (a soft poly ethylene glycol-terephalate block copolymer with a hard poly buthylene-terephthalate) among others.

Exemplary hydrophobic polymers include, without limiting, polycarbothane (aliphatic, polycarbonate-based TPU), Shore A 75 through Shore D 72, poly(Vinyl Acetate), polysulfone poly(vinyl chloride), biodegradable polylactide (PLA), polyethylene, polystyrene, polyvinylchloride, polytetrafluorethylene, polydimethylsiloxane, polyesters, polyurethanes, acrylics, epoxies, polylactic acid, polytetrafluoroethylene, polyketals, cellulose acetate. The polymeric fibers are electrospun fibers.

To achieve faster dissolution rates, the polymeric electrospun micro or nanofibers are highly hydrophilic in nature whereas highly hydrophobic fibers result in a reduced dissolution rate. In preferred embodiments, the dissolution rate ranges from about 1-5 minutes. In further embodiments, the nanofibers comprise a diameter ranging from about 1 nm to 1000 nm. In preferred embodiments, the nanofibers comprise a diameter ranging from about 0.05 μm to 0.5 μm. In preferred embodiments, the thickness (areal density) of the nanofiber is within a range of about 1-100 gsm. All ranges and subranges are contemplated to be within the subject matter of the present invention.

In some embodiments, the micro or nanofiber (that are substantially free of the active ingredient or devoid of active ingredients or cosmetic agents) is pre-wetted with water or optional cosmetically acceptable excipients, prior to applying on the skin. In alternative embodiments, the skin is wetted, or made moist with water or cosmetically acceptable excipients, prior to applying the nanofibers.

The applicant of the present invention unexpectedly and surprisingly achieved the benefit of improving the skin's appearance by using the composition disclosed herein. Applicants surprisingly found that even while the composition is substantially free of or devoid of active ingredients or cosmetic agents and in some instances, where no active ingredient or cosmetic agent is present, the nanofibers provided significant cosmetic benefit, including a reduction in the dilation of the veins, bags under the eyes, dark circles under the eyes, and swelling around the eyes, fine lines and wrinkles, loss of elasticity, wrinkles, loss of stiffness, loss of uniformity of color or tone, rough surface texture, age spots, and a decrease in moisture content in the skin around the eyes smoothing of the skin, fine lines and/or wrinkles, and lifting and firming of the skin.

The solid or semi-solid composition such as a film or a patch is completely dissolvable or disappearing. The film or patch may be partially dissolvable or disappearing. The composition of the present invention may be utilized with other modes of delivery including microneedles, iontophoresis, or electroporation. For example, in one embodiment, microneedles are applied to the skin and the composition disclosed herein, for example, without limiting, a film or a patch is placed on the top of the microneedles (which may be pre-treated to facilitate the dissolution of the patch). In another embodiment, the film or patch is part of an iontophoresis patch. Any and all combinations and permutations in the use are contemplated to be part of the present invention.

In some embodiments, the composition may be formulated for use more than one, two, three, four times a day. In preferred embodiments, the composition may be formulated for use twice a day. In more preferred embodiments, the composition may be formulated for use in the morning and prior to retiring to bed at night. In some embodiments, the composition may be formulated for use more than one, two, three, four times a day. In preferred embodiments, the composition may be formulated for use twice a day. In more preferred embodiments, the composition may be formulated for use in the morning and prior to retiring to bed at night. In preferred embodiments, the composition in form of the patch or the film may be applied prior to retiring to bed and may be allowed to stay on the skin throughout the night.

In one aspect, the composition of the present invention may be formulated as a solid or semi-solid cosmetic product, such as without limiting, a patch or a film.

In some embodiments, the composition can be formulated to have a pH in a range of about 1-10. In some embodiments, the compositions can be formulated to have a pH of about less than 3.0, 3.5, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, or more, or any range or integer derivable therein.

In one aspect, the present invention relates to a composition (e.g., a skincare composition) comprising a first layer under a second layer, said first layer comprising a biomaterial (including but not limited to a material selected from the group consisting of collagen, chitosan, cellulose, and alginate, as well as nanospheres thereof), said second layer comprising polymeric fibers, said fibers selected from the group consisting of microfibers or nanofibers. In a preferred embodiment, the second layer comprises electrospun microfibers (or nanofibers). In further embodiments, the polymeric microfibers (or nanofibers) comprise a hydrophilic polymer. In still further embodiments, the polymeric microfibers (or nanofibers) comprise a hydrophobic polymer. In additional embodiments, the polymeric microfibers (or nanofibers) possess adhesive properties upon wetting. In further embodiments, the polymeric microfibers (or nanofibers) form an invisible film upon wetting that further dissolves upon additional wetting. In still further embodiments, polymeric microfibers (or nanofibers) are substantially free of or devoid of active ingredient or agent. In some embodiments, the polymeric fibers may comprise cosmetically acceptable excipients or softeners. In some embodiments, one or more adhesive layers may be attached to the film or patch to ensure initial adhesive prior to wetting.

The composition according to the present invention is not limited by the nature of the polymer(s) used for the microfibers (or nanofibers). Any variety of polymers can be used. In some embodiments, multiple (different) polymers can be used together or separately. In a preferred embodiment, the composition is made using both hydrophobic and hydrophilic polymers. The present invention is also not limited by the nature of the biomaterial. In a preferred embodiment, the biomaterial is absorbent, and even highly absorbent. In such an embodiment, the present invention contemplates combining highly absorbent “sponges” with solid microfiber (or nanofiber) platforms to obtain advantages over other delivery technologies. The present invention is also not limited by the number of layers present in the composition. Single, double, triple-layer (or more are contemplated. For example, in one embodiment, a three-layer electrospun fiber is provided such that their layers are electrospun from different solutions via three tips on to one and same collector. In one embodiment, the present invention contemplates a multi-layer matrix, said matrix is substantially free of or devoid of active ingredients or cosmetic agents.

In one embodiment, the composition comprising polymeric fibers that are substantially free of or devoid of active ingredients is applied to the skin without pre-wetting the skin. In another embodiment, the composition comprising polymeric fibers that are substantially free of or devoid of the active ingredient is applied to the skin after pre-wetting the skin with a solution, such as water or a solvent that is free of the active ingredient or cosmetic agent.

In another aspect, a method of improving, treating, repairing, or reducing a skin's visual appearance is provided. The method comprises topically administering an effective amount of a composition comprising electrospun fibers that are substantially free or devoid of the active ingredient or cosmetic agents. The present invention relates to a method for repairing or improving the skin, comprising by providing a human subject, a skincare composition according to the above (e.g., polymeric microfibers or nanofibers that are substantially free of or devoid of active or cosmetic agents or ingredients) and administering the skin care composition by applying or contacting with the subject's skin. In some embodiments, the composition is a solid or semi-solid form such as a film or a patch. In further embodiments, the composition is administered topically or transdermally. In some embodiments, the composition may be applied more than one time in a given day. In some embodiments, the composition may be applied at night prior to retiring to bed. In some embodiments, the composition in the form of a patch or a film is applied prior to retiring to bed at night and is allowed to stay on the skin throughout the night.

In additional embodiments, the subject exhibits symptoms associated with or is suspected of having an affected visual appearance, physical properties or physiological functions of the skin, such as visually undesirable appearance to the skin, including dilation of the veins, bags under the eyes, dark circles under the eyes, and swelling around the eyes may occur, environmental damage to the skin, fine lines and wrinkles, loss of elasticity, wrinkles, loss of stiffness, loss of uniformity of color or tone, rough surface texture, age spots, and a decrease in moisture content, smoothing of the skin, fine lines and/or wrinkles, and lifting and firming of the skin. Topical application of the composition can aid or prevent such a skin condition. The effectiveness of the composition can be compared with skin that has not been addressed with a composition of the present invention. In certain non-limiting embodiments, the skin can be localized to and/or around an area (such as eyes) where the composition is applied to the skin. The skin can be a facial, torso, back, neck, ear, pelvic, arms, hands, legs (e.g., ankle, knee, thigh), feet, or buttocks skin. Non-limiting examples of skin defects that can be treated or prevented with compositions of the present invention include telangiectasia (i.e., spider veins), eye circles (e.g.,, dark circles under the eye), puffy eyes, pruritus, lentigo, age spots, senile purpura, keratosis, melasma, blotches, wrinkles, fine lines, nodules, sun-damaged skin, acne, or hyperpigmentation. In certain aspects, the skin condition can be caused by exposure to UV light, age, irradiation, chronic sun exposure, environmental pollutants, air pollution, wind, cold, heat, chemicals, disease pathologies, or smoking. The skin can be aged, nutritionally compromised, or environmentally damaged skin. In certain aspects, the composition can be topically applied in an amount effective to increase the stratum corneum turnover rate of the skin, collagen synthesis production of the skin, fat production of the skin, firmness of the skin, or elasticity of the skin. In other aspects, the composition can be topically applied in an amount effective to reduce or inhibit new capillary formation in or near the skin, blood flow to the skin, the fluid amount in or near the skin, or melanin production in the skin.

In some exemplary embodiments, the invention relates to a composition comprising electrospun polymeric microfibers (or nanofibers) that are substantially free of or devoid of active agents or ingredients. In some embodiments, the composition is made of PVP fibers or pullulan fibers, among others. When contacted with the skin, the PVP dissolves, or partially dissolves, thereby becoming completely or partially invisible to the naked eye. In preferred embodiments, the composition may comprise more than one layer. In some embodiments, the composition may be made of a combination of polymeric fibers that are known in the art. In further embodiments, the polymeric microfibers (or nanofibers) form an invisible film upon wetting that further dissolves completely or partially upon additional wetting. Again, it is not intended that the present invention be limited by the particular polymer used.

In other embodiments, the invention contemplates polymer fibers that are synthetically manufactured such as PCL, PLA, PLGA, PVOH, Poly Vinyl Acetate, PAN, PA, HA, PEO/HA, or mixtures thereof. In other embodiments, the invention contemplates natural polymer fibers such as pullulan or hyaluronic acid, or combinations thereof. In some other embodiments, the polymers can be ceramics-based such as oxides, carbides, nitrides, borides, or mixtures thereof, among others.

The present invention also contemplates systems comprising the various films or patches or semi-liquid composition described herein. Thus, in one embodiment, the present invention contemplates a system, comprising a pre-wetting solution, and a skin patch or a film, said skin patch comprising electrospun hydrophilic fibers (microfibers or nanofibers) and is substantially free of or devoid of active cosmetic agents or active ingredients. The pre-wetting solution is also free of or devoid of active ingredients or cosmetic agents. In one embodiment, the system further comprises instructions for pre-wetting skin and applying said patch under conditions to form an invisible film that dissolves completely in less than five minutes (e.g., less than 30 seconds, or more preferably between 1 and 20 seconds).

Also disclosed are kits or systems that can include a composition of the present invention. In certain non-limiting aspects, the composition is comprised in a container.

The container can be a bottle, dispenser, package, etc. The container can be configured to dispense a predetermined amount of the composition. The container can be configured to dispense the composition in a semi-solid, liquid, spray, emulsion, or aerosol form. In certain aspects, the kit or the system can include indicial on its surface and/or instructions for using the composition. In certain aspects, the kits or systems can include a prewetting solution that is substantially free of or devoid of active ingredients or agents, such as water or saline.

In other aspects of the present invention, the composition can be used as part of a regimen to treat a skin condition. For instance, the regimen can include applying a composition of the present invention in a first instance as disclosed throughout this specification. The regimen can then include additional applications that are identical, similar, or different than the first instance application. The additional applications can include, for example, a second, third, fourth, fifth, sixth, seventh, eighth, nine, tenth, or more applications with a composition of the present invention and/or whether another method for treating a particular skin condition (e.g., other compositions, methods, etc.). The regimen may also include applying the composition in the morning and/or prior to retiring to bed at night. The regimen may further include the application of the composition prior to retiring to bed and allowing the composition to stay on the skin throughout the night.

The compositions of the invention can accordingly be in the form of a patch or a mask comprising at least one layer of nanofiber (or microfiber). The nanofiber (or microfiber) may be hydrophobic or hydrophilic or mixtures thereof. The nanofiber (or microfiber) may be made from any material, including polymers, biomaterials, naturally occurring material, plant material, synthetic hydrophilic or hydrophobic polymers.

### EXPERIMENTS

### Example 1

A non-limiting clinical study to evaluate the effects of nanofiber patches according to the present invention was conducted. The study evaluated the effects of eye treatments with nanofiber patches with and without caffeine (active ingredient). The study also evaluated the appearance and properties of under-eye puffiness using in-life visual grading and 3D fringe imaging techniques. Under-eye lines were evaluated using in-life visual grading.

The study evaluated 25 panelists, within the age distribution of 40-49 years (2 panelists), 50-59 years (14 panelists), and 60-69 years (7 panelists). Treatment patches include 1. Nanofiber PVP patch; Nanofiber PVP patch with 20% caffeine and a serum.

The study inclusion criterion is listed below.
18-19 year-old females
Fitzpatrick skin type I-IV
Under eye puffiness grades 3-6
LG score distribution: [2,3]=3; [3,4]=7; [4,5]=8; [5,6]=3; [6,7]=2.

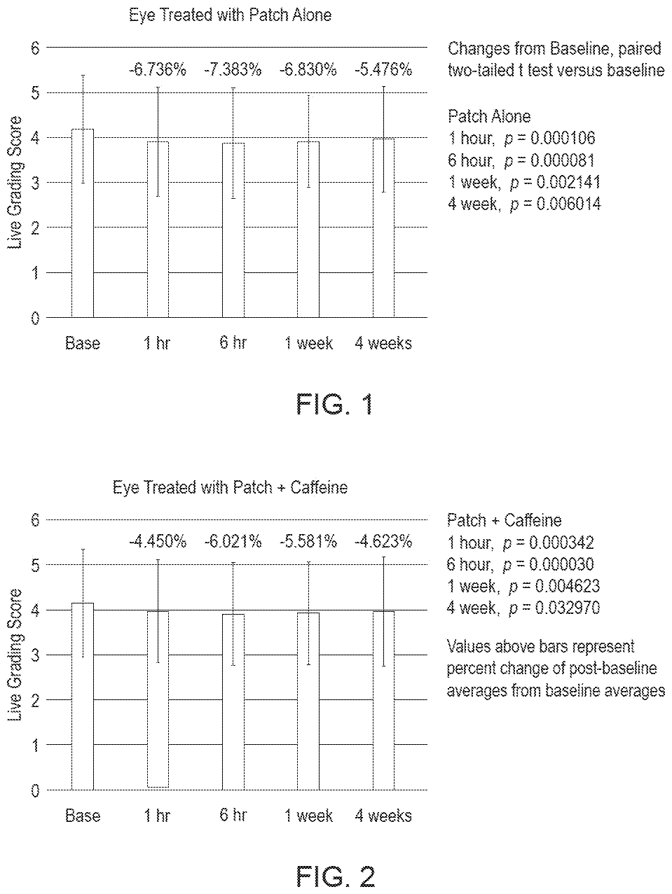

The patches were applied to designated eyes during P.M. Under-eye was wetted with serum and the patch was applied. The patch was allowed to reside on the skin for five minutes and the plastic backing/adhesive was removed. Patches were applied one eye at a time. The application was randomized to which side where the caffeine patch was applied. Treatments were performed using nanofiber PVP, nanofiber PVP with caffeine (20%), and serum alone without nanofiber. The study tested for under-eye puffiness, under-eye lines and wrinkles and visia cross-polarized standard 1 and 2, lighting conditions with R/C/L for each condition. The following time points were evaluated: baseline, one hour, six hours, one week, and 4 weeks. Post baseline evaluation was performed at 6 hours±30 minutes, 1 week±1 day, and 4 weeks±2 days. Results of live grading for under-eye puffiness, using nanofiber patches alone are shown in FIG. 1. Results of live grading for under-eye puffiness using nanofiber patches and caffeine are shown in FIG. 2.

Results show that there was a significant difference for patches over baseline at 1 hour, 6 hours, 1 week, and 4 weeks' time points. No significant difference was found between results from patches with or without caffeine. Further, nanofiber patches alone show a positive effect on reducing under-eye puffiness, and dark circles. This result was also confirmed via objective measurement through the PRIMOS study, laser profilometry (including, lifting and firming benefits) and additional in-vitro studies. It was also observed that the use of patches on the skin did not cause any adverse effect on the subject's skin and was very well tolerated.

### Example 2

An ex-vivo study was conducted to evaluate the impact of the firming effect of the nanofiber patches using MatTek skin (artificially wrinkled). The following material was tested: MatTek Skin; PVP original patch, thickness (areal density) 3 gsm; PVP patch, thickness (areal density) 3 gsm; PVP/HA patch, with actives, thickness (areal density) 3 gsm; HA/PEO, thickness (areal density) 3 gsm; and Pullulan, thickness (areal density) 10 gsm.

A respective patch circle was prepared by cutting the sheet with an 8 mm Disposable Biopsy Punch. The circle was drawn on the hydrated MatTek skin and the protective plastic was removed, while the nanomaterial dissolved into the skin. The LJ-V7080 was turned on and the samples were placed under the laser light beam. Computer parameters were implemented to obtain the picture via LJ Navigator App, Highlight Profile, Height Image, Measurement, Batch Start. Measurements were performed via LJ-Observer, Open Measurement Highlight Specify 2 pts, and open Profile1. Measured dimensions in topography profiles provided weight and height of the sample respectively. Results are shown in Table 1 below.

TABLE 1

| | Wrinkle | % | Wrinkle | % |
|---|---|---|---|---|
| Material | height(mm) | Reduction | Width(mm) | Reduction |
| MatTek | 0.307 | NA | 0.6 | NA |
| PVP original | 0.007 | 97.7 | 0.05 | 91.7 |
| patch | | | | |
| Thickness/areal | | | | |
| density 3 gsm | | | | |
| PVP patch | 0.05 | 83.3 | 0.4 | 33.3 |
| Thickness/areal | | | | |
| density 3 gsm | | | | |
| PVP/HA | 0.005 | 98.3 | 0.15 | 75.0 |
| Thickness/areal | | | | |
| density 3 gsm | | | | |
| HA/PEO | 0.4 | 33.3 | 0.25 | 16.7 |
| Thickness/areal | | | | |
| density 3 gsm | | | | |
| Pullulan | 0.0 | 100 | 0.0 | 100 |
| Thickness/areal | | | | |
| density 10 gsm | | | | |

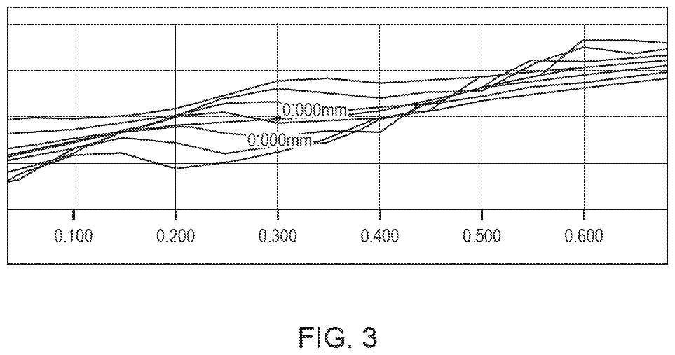

FIG. 3 shows the results of pullulan on MatTek skin. The measurements of the five nanofiber patches show differences in the MatTek skin wrinkle width and height reduction as compared with the wrinkled MatTek as a control. The results show that pullulan showed 100% wrinkle reduction, thereby showing improvement in repairing, reducing, and improving fine lines and wrinkles, elasticity, stiffness, uniformity, tightening of the skin, and rough surface texture, thereby improving the overall appearance of the skin, particularly around the eyes, smoothing of the skin, fine lines and/or wrinkles, and lifting and firming of the skin.

While particular embodiments of the present invention have been illustrated and described, it would be obvious to those skilled in the art that various other changes and modifications may be made without departing from the scope of the invention. It is therefore intended to cover in the appended claims all such changes and modifications that are within the scope of this invention.

What is claimed is:

1. A method of the reducing appearance of puffiness in periorbital area of a subject's skin, comprising topically applying a composition comprising electrospun fibers to the subject's skin in need thereof.

2. The method of claim 1, further causing firming or tightening of the skin around the periorbital area of the subject.

3. The method of claim 1, wherein the composition is substantially free of active ingredients.

4. The method of claim 1, wherein the composition is devoid of active ingredients.

5. The method of claim 1, wherein the composition further comprises cosmetically acceptable carriers.

6. The method of claim 1, wherein the composition further comprises an anti-oxidant or moisturizers or softeners or preservatives.

7. The method of claim 1, wherein the composition is a patch or a mask with a thickness ranging from about 0.1 to about 50 gsm.

8. The method of claim 1 wherein the electrospun fibers comprise hydrophilic polymers.

9. A method for improving the visual appearance of a subject's skin, comprising providing a subject a solution to wet the skin, and administering a composition topically to the skin, wherein the composition comprises electrospun fibers.

10. The method of claim 9, wherein the composition is substantially free of active ingredients.

11. The method of claim 9, wherein the composition is devoid of active ingredients.

12. The method of claim 9, wherein skin is wetted by spraying a solution or through a spray.

13. The method of claim 9, wherein the method improves dilation of the veins, bags under the eyes, dark circles under the eyes, and swelling around the eyes, lifting, firming, smoothing of the skin, fine lines, wrinkles, or tightening of the skin.

14. The method of claim 9, wherein the electronspun fibers comprise one or more hydrophilic polymers that dissolve in less than two minutes of wetting.

15. A cosmetic patch comprising hydrophilic electrospun fibers, wherein the patch is substantially free of active ingredients.

16. The cosmetic patch of claim 15 further comprises one or more adhesive layers.

17. The cosmetic patch of claim 15 is devoid of active ingredients.

18. A kit comprising a pre-wetting solution, and a cosmetic patch comprising hydrophilic electrospun polymeric fibers.

19. The kit according to claim 18, wherein the patch is a mask.

20. The kit according to claim 18, wherein the patch is a film.
